(19) United States
(12) Patent Application Publication
(10) Pub. No.: US 2005/0075501 A1
(43) Pub. Date: Apr. 7, 2005

(54) Ammonium compounds bearing and electrophilic fluorine, reagent containing same, method using same and synthesis method for obtaining them

(76) Inventors: Jean-Roger Desmurs, Saint-Symphorein (FR); Dominique Hebrault, Marblehead, MA (US); Dominique Cahard, Notre-Dame De Bondeville (FR); Christophe Audouard, Le Grand Quevilly (FR); Jean-Christophe Plaquevent, Notre-Dame De Bondeville (FR)

Correspondence Address: Kevin E McVeigh;Intellectual Property Department, Rhodia Inc CN 7500, 259 Prospect Plains Road, Cranbury, NJ, 08512-7500, US

(21) Appl. No.: 10/296,361

(22) PCT Filed: May 22, 2001
(86) PCT No.: PCT/FR01/01578

(30) Foreign Application Priority Data
May 23, 2000 (FR) 00/06557

(51) Int. Cl. C07D 453/04

(52) U.S. Cl. 546/134

(57) ABSTRACT

The invention concerns quaternary ammonium compounds whereof the nitrogen bears a fluorine atom. Said ammonium compounds have an asymmetric carbon atom which is spaced from the ammonium function by not more than five members. The invention is useful for enantioselective electrophilic fluorination of various substrates.

The subject of the present invention is novel electrophilic fluorinating agents. It relates more specifically to compounds containing a quaternary ammonium functional group whose nitrogen bears a fluorine atom, and the use of these quaternary ammonium molecules for forming a reagent capable of carrying out enantioselective electrophilic fluorinations (EEF).

In the remainder of the description, we will adhere to the practice according to which chemical compounds are designated by a major functional group considered, which functional group then becomes the eponym of the compounds considered.

Thus, the compounds bearing the quaternary ammonium functional group targeted in the present invention will be designated by “quaternary ammonium compound”.

The fluorination of organic compounds has always been a delicate problem because of the specificities of the fluorine atom and molecule. The fluorine molecule exhibits such a reactivity that direct fluorinations are practically impossible, except for a few families of very specific compounds.

Accordingly, the procedure is generally carried out by the indirect routes, either by means of cobalt(III) fluorides, or by chlorination, or more generally hydrogenation or chlorine-fluorine exchange.

The problem is even more acute when it is desired to obtain fluorinated compounds of the atom bearing the fluorine exhibiting chirality.

The problem is even more acute since the introduction of fluorine is carried out late in the synthesis of the desired compound.

However, over the past fifteen years, fluorinated compounds have assumed increasing importance in molecules with biological, in particular pharmaceutical and agrochemical activity.

Accordingly, the market is in need of fluorinating reagents and agents which avoid carrying out the fluorination in several steps and which do not have an excessive cost.

Electrophilic fluorinations have the advantage of requiring in general only few steps.

However, there are very few reagents which are of interest and which have a broad efficacy range in the field of enantioselective fluorination.

As a reminder, there may be mentioned the work by E. Differding Tetrahedron Letters, 29, 1988, 6087-6090 who proposed using fluorosultames for carrying out enantioselective fluorinations. This technique only gives good results for few substrates and hardly involves malonic type derivatives. In other cases, the yields and the enantiomeric excess remain very low.

These results have been slightly improved by F. A. Davis (J. Org. Chem., 63, 1998, 2273-2280), but the sultames remain compounds which are difficult to obtain and which are particularly expensive.

In parallel with these studies, some authors (see the article by R. Banks in Journal of Fluorine Chemistry, 87 [1998] 1-17) have shown that certain DABCO (diazabicyclooctane) derivatives could be fluorinated to give one of the fluorinated quaternary nitrogen compounds and that such a reagent could serve as fluorinating agent in some cases.

However, this technique does not lead to an enantioselective fluorination.

These fluorinated compounds of DABCO are currently marketed under the trade mark Selectfluor.

Accordingly, one of the aims of the present invention is to provide a novel fluorinating agent which is capable of giving good fluorination yields.

Another aim of the present invention is to provide a fluorinating agent of the above type which is capable of giving enantioselective fluorinations with a significant enantiomeric excess.

Another aim of the present invention is to provide a reagent using such a fluorinating agent; another aim of the present invention is to provide a method for synthesizing the fluorinating agents according to the invention.

Finally, another aim of the present invention is to provide a method for using the fluorinating reagent in order to give good yields and a good enantiomeric excess (ee).

These aims, and others which will subsequently emerge, are achieved by means of a quaternary ammonium compound having at least one asymmetric atom and whose quaternary ammonium functional group bears a fluorine and is distant by the shortest route by only at most four members, advantageously at most three members, preferably at most two members, from said asymmetric atom (the nearest when there are more than one thereof). The expression members is understood to mean divalent atoms or groups in which the number of members includes neither the asymmetric carbon considered, nor the nitrogen quaternized by the fluorine.

Thus, if the product exemplified in the present application is considered, namely N-fluorocinchonidinium, two asymmetric atoms satisfy condition 1 which is directly linked to the quaternary nitrogen and the other which is linked to the quaternary nitrogen by a member, which member is the preceding chiral carbon.

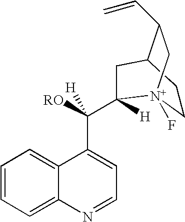

In this formula, it can be seen that several routes may be taken in order to go from the quaternary nitrogen atom to the various chiral atoms. The route to be taken into account is that which comprises the fewest members.

The alcohol functional group may be protected (ether, ester and the like).

The general formula of the preferred compound according to the invention may be written as

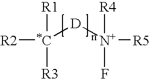

where R₁, R₂ and R₃ are different and are chosen from hydrogen, halogens or the functional groups below:
-alcohol;
amine;
amide;
thiol;
   where R₄ and R₅ are chosen from hydrocarbon derivatives

where D represent divalent linkages providing the bond between the chiral carbon and the fluorinated quaternary ammonium compound

where n represents an integer at most equal to 4, advantageously to 3, preferably to 2 and advantageously at least equal to 1;

the optional D, which may be identical or different are advantageously chosen from chalcogens, optionally substituted methylenes, and group VB metalloid atoms such as nitrogen, (the periodic table of elements used in the present application is that of the supplement to the Bulletin de la Société Chimique de France, January 1966, No. 1).

Given the instability which they would confer on the molecules, it is desirable that when D represents two chalcogens, especially if they are identical, the chalcogens should be separated by at least one carbon, preferably two.

It is preferable that the Ds are all chosen from optionally substituted methylenes, and oxygen, for at most one of them. It is preferable that R₁ is a small-sized group, advantageously methyl, or a single-atom group such as halogen and especially hydrogen.

Advantageously, R₂ represents a hydrocarbon radical, that is to say containing carbon, and hydrogen or an alcohol functional group, in particular R₂ may be alkyl, including aralkyl, aryl, including alkylaryl, alkyloxy, including aralkyloxy, aryloxy, including alkylaryloxy. R₂ is also preferably a radical obtained from the esterification of an organic or inorganic acid with the alcohol functional group, in particular an acyloxy radical. The esterifications with aromatic acids (that is to say acids in which the functional group bearing the acidity is directly linked to an aromatic ring such as benzoic or arenesulfonic acids), especially aromatic carboxylic acids give very good results.

R₃ is advantageously a hydrocarbon, advantageously aromatic, radical often low in electrons; such as nitrobenzenes or pyridine, including quinoline, rings.

In the case of N-fluorocinchonidinium, the compounds corresponding to the formula:

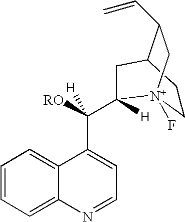

N-fluorocinchonidinium are among the most active.

With chosen from aromatic and aliphatic acyls, alkyls and aryls.

According to the present invention, the ammonium compounds are preferably such that the asymmetric atom, or at least one of them, is a carbon atom.

It is preferable that this asymmetric atom bears a nonoxidizable hydrogenophore functional group. The expression hydrogenophore should be understood to mean bearing hydrogen and the expression nonoxidizable should be understood to mean that this functional group is not capable of being oxidized by the quaternary ammonium functional group bearing a fluorine.

These hydrogenophore functional groups may be in particular thiol functional groups, amine functional groups, amide functional groups or alcohol functional groups. However, the thiol functional groups risk being too oxidizable, the amine functional groups and the amide functional groups are capable of interfering during the reaction, especially when bases are used; accordingly, alcohol functional groups are preferred.

The alcohol functional groups, in particular acylated or etherified alcohol functional groups, also give good results.

The quaternary ammonium functional group, in addition to the fluorine atom, is advantageously linked to carbon atoms, advantageously all those of the sp³ hybridization.

As has been seen above, said asymmetric atom, or one of the asymmetric atoms, bears a hydrogenophore functional group capable of giving rise to hydrogen bonds and promotes ipso facto the chiral induction. Other groups may also improve the chiral induction, alone or in combination with said hydrogenophore function. They are in particular radicals of groups bearing an aromatic ring, often low in electrons, such as nitrobenzenes or pyridine, including quinoline rings. The latter radical is advantageously distinct from the other three and is often methyl, or preferably hydrogen.

Thus, according to a preferred embodiment of the present invention, the chiral atom considered contains, as substituent:

an arm linking the quaternary ammonium functional group;

a hydrogenophore functional group;

a group bearing an arylic functional group, in general an aryl or an aralkyl, optionally substituted;

an advantageously small atom or group such as hydrogen and methyl.

According to one of the preferred embodiments of the present invention, in particular when the asymmetric carbon considered bears a hydrogen, it may be advantageous to protect the hydrogenophore functional group (alcohol, amide, or even thiol and amine), which then reduces the risk of oxidation by maintaining the possibility of a strong chiral induction as a radical ensuring the protection, there may be mentioned alkyls, acyls, sulfonyls which are aromatic or aliphatic and the customary protecting groups known in alkaloid, peptide and nucleic acid chemistry. The chiral inductions derived from the alcohols protected by alkyls and by acyls are remarkable. The alkyls (taken in the etymological sense of an alcohol from which the OH functional group has been removed), including aralkyls, advantageously have at most 10 carbon atoms and preferably at most 5 atoms, more preferably 3.

In the context of the above embodiment of the present invention, preferences may be expressed on the asymmetric carbon or one of the asymmetric carbons as below, the chiral atom considered contains as a substituent

an arm linking it to the quaternary ammonium functional group;

a protected hydrogenophore functional group, advantageously an alcohol functional group protected in the form of an ether or an ester;

a group bearing an aryl functional group, in general an aryl or an aralkyl, which is optionally substituted;

an atom or a group which is advantageously small, such as hydrogen and methyl.

The quaternary ammonium functional group is advantageously intracyclic, that is to say that it constitutes the member of a ring, which ring is advantageously not aromatic. This functional group is in general chiral.

According to the present invention, it is preferred that the quaternary ammonium functional group constitutes the member of at least two rings.

It is desirable that the counter-ion of the quaternary ammonium compound is an anion which is not very polarizing and is a poor nucleophile.

These properties are often correlated with the strength of the acid associated with the anion; accordingly, counter-ions whose associated acid has a pKa at most equal to two, advantageously to one or preferably to zero, are preferred.

This type of counterions is also encountered in the anions comprising several atoms and which can therefore be described as being polyatomic. In particular, there may be mentioned complex anions based on fluorine, such as PF₄⁻, PF₆⁻. There may also be mentioned the first acidity of sulfuric acid, chlorates and perchlorates, organic acids perfluorinated on the carbon bearing the acid functional group, such as perfluoroalkanoic acids and sulfonic acids bearing a perfluorinated carbon such as for example triflic acid. The imides corresponding to such acids perfluorinated on the carbon bearing the sulfonic functional group also give very good results. Triflimide may be mentioned as an example of such imines.

In general, the acids which correspond to the anions used as electrolytes in nonaqueous media and in batteries, in particular lithium batteries, generally give good results as counter-ions of the quaternary ammonium compounds according to the present invention.

The preferred anions, or at least the most widely used, are the triflates, the fluoborates and the PF₆⁻ ions. The anions corresponding to monoacids are preferred although the diacids, such as for example fluosilicic acid, may be used.

As was mentioned above, another aim of the present invention is to provide an electrophilic fluorinating reagent using the ammonium compounds according to the present invention.

Thus, the present invention relates to a reagent containing an ammonium compound as described above, that is to say a reagent containing a quaternary ammonium compound having at least one asymmetric atom and whose eponymous functional group bears a fluorine and is situated at most four members from said asymmetric carbon.

According to the present invention, said reagent contains, in addition, an advantageously aprotic solvent. It is also preferable that this solvent is polar.

As will be seen later, taking into account the fact that in order to obtain good enantiomeric excess it is desirable to work at low temperature, it is advisable to choose, as solvent, those which have a very low melting point. Thus, it is preferable that this solvent has a melting point at most equal to 10° C., preferably to −20° C., more preferably to −40° C.

Among the solvents giving good results, ethers, in particular cyclic ethers, nitrites and even aromatic derivatives may be mentioned.

The preferred derivatives are the cyclic ethers and the nitrites.

The reagent according to the present invention often contains, for successive or simultaneous addition, apart from the ammonium according to the present invention and optionally the solvent, a base. Advantageously, the associated acid of the base has a pKa of at least 16, preferably of at least 18.

The nature of the base is not unimportant for the yield linked to the enantiomeric excess. To obtain good yields and good enantiomeric excess, it is desirable that the cation of the base is not too small. It is thus preferable to use alkali metal cations of a higher rank than that of lithium. The Schwesinger type bases also give good results (cf. Tetrahedron Letters 39, [1998], 8775-8778).

It is also possible to use, in addition, alkali metals, oniums (quaternary ammonium compounds and phosphonium compounds which are trialkylated). According to the present invention, it has also been shown that it is preferable that the base, or more exactly the anion of the base, does not remain in the reaction medium and, for example, is volatile.

Thus, it has been shown that hydrides, which after removal of a proton, are released in the form of hydrogen, give better results than most of the other bases.

Although nonvolatile, alkali metal salts of hexamethyldisilazane give good results, especially in terms of enantiomeric excess.

The base may also be produced in situ, for example by the action of a fluoride ion on silylated derivatives. The quantity of base may be of some importance and the reagent may contain it up to twice, or even more, expressed as equivalents of said quaternary ammonium compound.

The excess of base improves yields, but only slightly modifies the enantiomeric excess.

The subject of the present invention is also to provide a method for the synthesis of fluorinated ammonium compounds according to the present invention. These compounds may in particular be produced by the action of a reagent reputed to act as “F⁺”, but according to a preferred embodiment of the present invention, these compounds are produced by putting the corresponding amine with the fluorinating agent designated by Selectfluor and often designated by the abbreviation F-TEDA-BF₄⁻. The reaction is advantageously carried out in a solvent and at room temperature. Acetonitrile gives good results as solvent.

According to a preferred embodiment of the present invention, the amine which, by fluorination, will give the ammonium compound according to the present invention in an equimolar quantity is subjected with said Selectfluor in a solvent, in general acetonitrile. The mixture is kept stirred for ¼ h to 5 h and then the solvent is evaporated by means of a rotary evaporator. The amine derived from the Selectfluor is precipitated by means of a strong acid (often sulfuric acid) after filtration and removal of the salt, precipitation is carried out by means of a third solvent from an acetone solution. The filtration makes it possible to obtain the desired compound. This procedure makes it possible to obtain ammonium compounds according to the present invention derived from cinchona alkaloids; in particular, N-fluorocinchonididium, N-fluorocinchoninium, N-fluoroquininium and N-fluoroquinidinium were thus obtained. The yields obtained are excellent, especially for the first two members of the family cited. Another aim of the present invention is to provide a method of electrophilic fluorination which may be stereoselective using the reagents of the present invention.

According to the present invention, this aim is achieved by mixing the reagent as defined above with the substrate and allowing the reaction to proceed for ¼ h to 24 h at temperatures which may range from −100° C. to 50° C.

The enantiomeric excess depends on the temperature and it is often advisable to work at a temperature of less than −20° C., preferably at most equal to 30° C.

It is possible to work in particular at −78° C., a temperature which is easy to obtain using dry ice.

The substrates which are capable of giving good results are molecules having an active hydrogen on a prochiral or even chiral carbon.

The hydrogen is made active by the presence of electron-attracting groups carried by the chiral or prochiral carbon. It is considered in the present description that an electron-attracting group is a group having a positive σₚ and advantageously greater than 0.5, more preferably greater than 0.10. The best results are obtained with values greater than 30. Halogen atoms are not included in these electron-attracting groups. Among the preferred attracting groups, there may be mentioned in particular trifluoromethyl groups, and more preferably carbonyl and nitrile groups.

Compounds of a malonic nature give acceptable results.

For reasons of volume yield, it is preferable that the ammonium compound according to the present invention has less than 50 carbon atoms, preferably at most 30.

The same applies to the substrates.

Another category of substrates gives good results; they are the double bonds rich in electrons such as, for example, enol ethers or enol esters. A particular mention should be made of silyl and enol ethers.

In the case of the enol derivatives, the base is still not necessary.

The following nonlimiting examples illustrate the invention.

General Points

The apparatus used during all the analyses are the following:

infrared: IR-FT Perkin Elmer 16 PC spectrophotometer

¹H NMR(300 MHz), ¹³C(75 MHz), ¹⁹F(282 MHz).

Polarimetry: Perkin Elmer 241 PE polarimeter.

GC on a chiral column (Supelco-β-dex).

### EXAMPLE 1

### Synthesis of the Ammonium Compounds According to the Present Invention: Synthesis of N-fluoroquinium Tetrafluoroborate

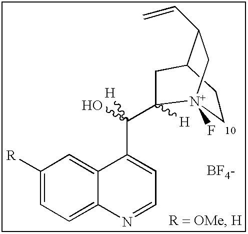

The starting alkaloid (13.6 mmol) in solution in 50 ml of HPLC grade acetonitrile is placed in a 100 ml two-necked round-bottomed flask and the mixture is vigorously stirred because of the low miscibility of the alkaloid in this solvent. The Selectfluor™ (13.6 mmol, 1 equivalent) is added by a funnel for solids over 15 min. The reaction mixture decolorizes completely and becomes clear. It is stirred for an additional 2 h at room temperature. Next, the acetonitrile is evaporated using a rotary evaporator and coevaporation with acetone: a foam forms in the round-bottomed flask at the end of the evaporation. This foam is redissolved in acetone and there is added dropwise an acetone solution containing one equivalent of concentrated H₂SO₄ at 96% by a dropwise dropping funnel: a white precipitate forms in the mixture. The solution is filtered and pure 1-chloromethyl-4-aza-1-azoniabicyclo[2.2.2]octane tetrafluoroborate is then obtained. There is then the amine sulfate corresponding to the starting material which is removed. About 250 ml of ether are added until a white precipitate forms. Filtration makes it possible to recover the desired compound in the form of a white solid with a yield of 98%. The same procedure makes it possible to obtain various ammonium compounds according to the present invention.

| Fluorinating agent (tetrafluoroborate) | Yield % |
|---|---|
| N-fluorocinchonidinium | 98 |
| N-fluorocinchoninium | 92 |
| N-fluoroquininium | 67 |
| N-fluoroquinidinium | 62 |

Analysis of N-fluorocinchonidinium Tetrafluoroborate:

¹H NMR (300 MHz, CD₃CN): 9.08 (d, J=5.4 Hz, 1H); 8.80 (bs, 1H); 8.30-7.95 (m, 5H); 6.61 (s, 1H); 5.78 (m, 1H); 5.23 (s, 1H); 5.16 (m, 1H); 5.00 (m, 1H); 4.48 (m, 2H); 4.38 (m, 1H); 3.45 (m, 2H); 3.24 (m, 1H). 2.62 (m, 2H); 2.20 (m, 2H); 2.19 (m, 1H); 1.98 (m, 1H).

¹³C NMR (75 MHz, CD₃CN): 153.31; 147.22; 141.62; 136.50; 134.36; 130.88; 125.93; 125.12; 124.40; 120.98; 118.49; 74.40 (d, J=9.4 Hz); 68.58 (d, J=9.2 Hz); 63.75 (d, J=4.7 Hz); 59.6.2 (d, 9.2 Hz); 43.36 (d, J=3.5 Hz); 28.58 (d, J=4.7 Hz); 27.40 (d, J=4.9 Hz); 23.90.

¹⁹F NMR (282 MHz, CD₃CN/CCl₃F): 37.71 (s, 1F); −154.52 (s, 1F); −154.60 (s, 3F). (CD₃CN/TFA): 118.35 (s, 1F); −74.12 (s, 1F); −74.17 (s, 3F). FTIR: (ν N—F) 927 cm^{-1 Melting point:} 189+/−2° C. Solubility: acetonitrile (ca. 200 mg/ml), water (very soluble), acetone (very sparingly soluble). MS (FAB⁺, glycerol) 313 (cation⁺, 18%) 295 (cation-H₂O, 100) . TLC: acetone/heptane: 9/1; Rf=0.3.

### EXAMPLE 2

### Synthesis of the Silylated Enol Ether of 2-methyl-1-tetralone

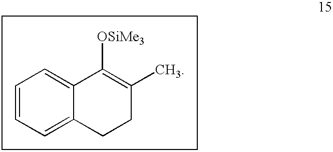

2-Methyl-1-tetralone (0.75 g, 710 ml, 4.68 mmol, 1 equivalent), NEt₃ (0.592 g, 813 ml, 5.851 mmol, 1.2 equivalent), TMSCl (0.636 g, 743 ml, 5.851 mmol, 1.2 equivalent) are placed successively in a 5 ml round-bottomed flask, a clear yellow solution is obtained. NaI (0.878 g, 5.851 mmol, 1.2 equivalent) in solution in 6 ml of acetonitrile is slowly added: a brown precipitate forms. The mixture is stirred for 4 h at room temperature: a brown liquid is then obtained with a lot of white insolubles in suspension. The mixture is filtered so as to obtain a white solid and a brown filtrate. The filtrate is extracted with three times 10 ml of pentane. The pentane phase is very light clear yellow. Flash chromatography on silica gel (eluent 90% heptane/10% ether) makes it possible to obtain, after evaporation of the solvents, a pale yellow viscous liquid with a yield of 94% is obtained.

Analysis:

¹H NMR (300 MHz, CDCl₃): 7.40 (d, J=7.4 Hz, 1H); 7.26 (m, 1H); 7.17 (t, J=2.7 Hz, 2H); 2.82 (t, J=7.5 Hz, 2H); 2.34 (t, J=8 Hz, 2H); 1.9 (s, 3H); 0.29 (s, 9H).

¹³C NMR (75 MHz, CD₃CN): 142.83 (Cq), 136.35 (CH), 134.78 (Cq), 127.13 (CH), 126.59 (Cq), 126.48 (CH), 121.91 (Cq), 117.35 (CH), 29.52 (CH₂), 28.68 (CH₂), 17.77 (CH₃), 1.02 (CH₃).

### EXAMPLE 3

### Fluorination of 2-methyl-1-tetralone

1. Passing by its Enolate

2 equivalents of NaH at 95% (20.2 mg, 0.8 mmol) in solution at room temperature under an argon atmosphere in 1 ml of anhydrous THF are placed in a 5 ml round-bottomed flask. The suspension is stirred for 10 min and then 2-methyl-1-tetralone (0.33 mmol, 52.8 mg, 1 equivalent) is added dropwise still at room temperature; it is then seen that the reactibn mixture changes color with the formation of the enolate. The temperature of the reaction mixture is reduced to −40° C. and the round-bottomed flask is allowed to stand for 10 min, with stirring. N-fluorocinchonidinium tetrafluoroborate (160 mg, 0.4 mmol, 1.2 equivalent) in solution in 1 ml of acetonitrile is added with a propelled syringe over 2 h: the reaction mixture changes in appearance. The reaction is stopped by hydrolyzing with 2 ml of water. The products are extracted with three times 10 ml of ether and then dried over MgSO₄. The purification is carried out by chromatography on silica gel (eluent: heptane/ether: 9/1) giving 2-fluoro-2-methyl-1-tetralone with a yield of 94%. The enantiomeric excess measured by GC is 56%.

2. From the Silylated Enol Ether

N-Fluorocinchonidinium Tetrafluoroborate (89.6 mg, 0.224 mmol, 1 equivalent) in suspension in 1 ml of THF is placed added to a 5 ml round-bottomed flask and the temperature is reduced to −78° C. The silylated enol ether of 2-methyl-1-tetralone (40 mg, 46 μl) in solution in 1 ml of THF is added over 2 h using a propelled syringe. After 24 h, the reaction is stopped by hydrolyzing with 2 ml of 2N HCl . The products are extracted with three times 10 ml of ether and then dried over MgSO₄. The purification is carried out by chromatography on silica gel (eluent:heptane/ether: 9/1) giving 2-fluoro-2-methyl-1-tetralone with a yield of 51%. The enantiomeric excess measured by GC is 92%.
Analysis:

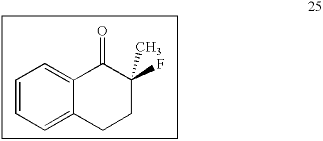

¹H NMR (300 MHz, CDCl₃): 8.00 (t, J=2 Hz, 1H); 7.40 (h, J=2.5 Hz, 1H); 7.21 (m, 2H); 2.98 (m, 2H); 2.20 (m, 2H); 1.55 (d, J=22 Hz, 3H).

¹⁹F NMR (282 MHz, CDCl₃/TFA): 9.81 (1F).

TLC: Et₂O/heptane: 1/1; Rf=0.4.

Chiral GC: temperature program: initial temperature 65° C., 2 min at 65° C., and then 5°/min up to 170° C., 2-methyl-1-tetralone: 20.70, 2-fluoro-2-methyl-1-tetralone: 21.05 and 21.40.

### EXAMPLE 4

### Fluorination of 2,2,6-trimethylcyclo-hexanone

Procedure Identical to 4-c-1. 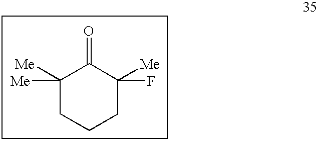
Analysis:

Chiral GC: temperature program: initial temperature 65° C., 2 min at 65° C., and then 5°/min up to 170° C., 2-fluoro-2,2,6-trimethylcyclohexanone: 7.50 and 7.75, 2,2,6-trimethylcyclohexanone: 8.69 and 8.95.

### EXAMPLE 5

### Fluorination of Ethyl Cyclopentanone-2-carboxylate

Procedure Identical to 4-c-1. 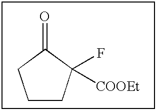
Analysis

¹H NMR (300 MHz, CDCl₃): 4.33 (q, J=7 Hz, 2H); 2.55 (t, J=7 Hz, 2H); 2.34 (m, 2H); 2.19 (q, J=7.5 Hz, 2H); 1.36 (t, J=7 Hz, 3H).

¹⁹F NMR (282 MHz, CDCl₃/TFA): −164.54 (t, 1F). TLC: Et₂O/heptane: 1/1; Rf=0.5. Chiral GC: temperature program: initial temperature 65° C., 2 min at 65° C., and then 5°/min up to −170° C., ethyl 2-fluorocyclopentanone-2-carboxylate: 14.70 and 14.85, ethyl cyclopentanone-2-carboxylate: 14.91.

### EXAMPLE 6

### Fluorination of an Alpha-Amino Acid

A solution of N-phthaloyl-2-phenyl-glycidonitrile (0.1 mmol, 26.24 mg) in 2 ml of THF cooled to −78° C., a N solution in Ir THF of Li HMDS (1.5 equiv., 0.15 mmol) is added dropwise. After stirring for 15 minutes under the same conditions, there are then added in a single portion 1.1 equivalent of the fluorinating agent, namely 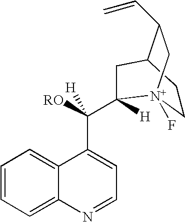
with R representing paramethoxybenzoyl.

The reaction is stopped after two hours by addition of a saturated aqueous ammonium chloride solution. After having expelled the solvents, the mixture is taken up in ether and subjected to preparative chromatography. A yield of about 93% and an enantiomeric excess of 94% are obtained.

1-23. (canceled)

24. A quaternary ammonium compound having at least one asymmetric atom whose quaternary ammonium functional group bears a fluorine atom and is distant from said asymmetric atom by the shortest route by only at most 4 members.

25. The quaternary ammonium according to claim 24, wherein the quaternary ammonium functional group bears a fluorine atom and is distant from said asymmetric atom by the shortest route at most 2 members.

26. The ammonium compound as claimed in claim 25, wherein the asymmetric atom is a carbon atom.

27. The ammonium compound as claimed in claim 24, wherein the asymmetric atom is a carbon atom bearing a nonoxidizable hydrogenophore functional group.

28. The ammonium compound as claimed in claim 24, wherein the asymmetric atom is a carbon atom bearing a hydroxyl functional group.

29. The ammonium compound as claimed in claim 24, wherein the asymmetric atom is a carbon atom bearing a protected hydrogenophore.

30. The ammonium compound as claimed in claim 24, wherein the asymmetric atom is a carbon atom bearing a hydroxyl functional group, protected in the form of an ether or of an ester.

31. The ammonium compound as claimed in claim 24, wherein the asymmetric atom is a carbon atom bearing an aryl or aralkyl radical.

32. The ammonium compound as claimed in claim 24, wherein the nitrogen of the ammonium functional group bearing the fluorine is intracyclic.

33. The ammonium compound as claimed in claims 32, wherein the nitrogen of the ammonium functional group bearing the fluorine is intracyclic with at least two rings.

34. The ammonium compound as claimed in claim 24, wherein the asymmetric carbon atom is linked to both a radical bearing the fluorinated ammonium functional group, to a radical, distinct from the preceding one, bearing an aromatic ring, and to a hydrogenophore functional group, advantageously to an alcohol functional group.

35. The ammonium compound as claimed in claims 24, wherein said compound is further combined with a counter-ion which is nonpolarizing and a poor nucleophile.

36. The ammonium compound as claimed in claim 35, wherein said counter-ion presents an associated acid having a pKa at most equal to 2.

37. The ammonium compound as claimed in claim 24, further combined with a polyatomic counter-ion.

38. The ammonium compound as claimed in claim 24, further combined with a fluorinated counter-ion.

39. A reagent useful for electrophilic fluorination, comprising a quaternary ammonium compound as claimed in claim 24.

40. The reagent as claimed in claim 39, further comprising a polar aprotic solvent.

41. The reagent as claimed in claim 39, further comprising a base.

42. The reagent comprising a quaternary ammonium compound as claimed in claim 36, further comprising a base and an associated acid having a pKa at least equal to 18.

43. The reagent as claimed in claim 42, wherein the base is generated in situ, by a fluoride ion reacting on an alcohol or a silylated amine.

44. A process for the preparation of a quaternary ammonium compound having at least one asymmetric atom whose quaternary ammonium functional group bears a fluorine atom and is distant from said asymmetric atom by the shortest route by only at most 4 members, said process comprising the step of bringing the amine corresponding to said quaternary ammonium compound into contact, in a polar solvent, with an N-fluoro-1,4-diazoniabicyclooctane in the form of a dissociated salt.

45. A method of fluorination, comprising the step of reacting a reagent as defined in claim 39, into contact with a substrate rich in electrons.

46. The method as claimed in claim 45, wherein the reaction is carried out at a temperature of at most 20° C.

47. A method of fluorination as claimed in claim 45, wherein said fluorination is an enantioselective electrophilic fluorination.
